(19) United States
(12) Patent Application Publication
(10) Pub. No.: US 2023/0233641 A1
(43) Pub. Date: Jul. 27, 2023

(54) ORAL GEL COMPOSITIONS

(71) Applicant: Colgate-Palmolive Company, New York, NY (US)

(72) Inventors: Bindu AIYAR, Mumbai (IN); Purushottam JADHAV, Mumbai (IN); Payal ARORA, Lebanon, NJ (US); Kshitij SATARDEKAR, Mumbai (IN); Nikhil MALI, Mumbai (IN)

(73) Assignee: Colgate-Palmolive Company, New York, NY (US)

(21) Appl. No.: 18/093,654

(22) Filed: Jan. 5, 2023

(30) Foreign Application Priority Data
Jan. 7, 2022 (IN) 202211000994

(51) Int. Cl. A61K 36/61; A61K 9/00; A61K 36/54; A61P 29/00; A61K 45/06; A61K 47/10; A61K 36/9066

(52) U.S. Cl. CPC A61K 36/61; A61K 9/0053; A61K 36/54; A61K 36/9066; A61K 45/06; A61K 47/10; A61P 29/00; A61K 36/484

(57) ABSTRACT

Disclosed herein are orally acceptable topical analgesic gels comprising a mixture of herbal oils and extracts comprising (a) clove oil and/or eugenol (e.g., clove leaf oil and/or clove bud oil), (b) a cooling agent (e.g., menthol), (c) camphor, (d) an antimicrobial agent, e.g., turmeric and/or curcumin extract (e.g., turmeric, white turmeric (e.g., white curcumin) and (e) licorice; in an orally acceptable gel base, the gel base comprising one or more nonionic surfactants, the formulation providing a sustained release of the mixture of herbal oils and extracts following application to a tooth or gums; together with methods of making and using the same.

### FIELD OF THE INVENTION

Disclosed herein are orally acceptable topical analgesic gels comprising a mixture of herbal oils and extracts comprising (a) clove oil and/or eugenol (e.g., clove leaf oil and/or clove bud oil), (b) a cooling agent (e.g., menthol), (c) camphor, (d) an antimicrobial agent, e.g., turmeric and/or curcumin extract (e.g., turmeric, white turmeric (e.g., white curcumin) and (e) licorice; in an orally acceptable gel base, the gel base comprising one or more nonionic surfactants, the formulation providing a sustained release of the mixture of herbal oils and extracts following application to a tooth or gums; together with methods of making and using the same.

### BACKGROUND

Bacteria in the mouth can form plaque on the teeth. These bacteria may also cause an individual’s gums to become inflamed. If not addressed, this can further result in red, swollen, or bleeding gums. In various cases this can lead to gingivitis and the untreated gum inflammation and irritation can result in significant amounts of pain. If left untreated, gingivitis can worsen and ultimately lead to tooth loss.

Moreover, tooth pain is most often caused by structural damage to the tooth, wherein the nerves of the tooth, which are normally well protected by dentin and enamel, is exposed to external stimuli, for example as a result of caries, a cracked tooth, an exposed tooth root, or erosion of the enamel, as well as by gum disease, abscess, or impaction. The severity of a toothache can range from chronic and mild to sharp and excruciating. The pain may be aggravated by chewing or by cold or heat or moving air.

The patient may not be able to identify the cause of the gum or tooth pain without a dental examination. Current symptomatic treatments include pharmaceutical analgesics - non-steroidal anti-inflammatory agents such as such as aspirin, ibuprofen, or acetaminophen, topical gel anesthetics containing lidocaine or benzocaine, and/or narcotics such as codeine - but these pharmaceuticals each have their own limitations, in that they may not be available without a prescription, may not have an immediate effect, and/or may have undesirable side effects. Home remedies may provide a brief respite from the pain, but do not remain for extended periods on the tooth and typically do not provide controlled delivery of the active ingredient or sustained pain relief.

There is a need for improved methods of treating and/or soothing gum and/or tooth pain, which address the various types of pain, for example pain from cavities as well as from dental hypersensitivity, gum inflammation and gum irritation and which provide immediate and sustained relief from the pain.

### BRIEF SUMMARY

The disclosure provides, in a first aspect, a novel topical “leave on” gel formulation that permits sustained delivery of pain-relieving herbal extract ingredients to the affected tooth or gum area. In one aspect, the disclosure provides leave-on gel formulations comprising a unique blend of oils and/or extracts (e.g., soothing oils or extracts) comprising: clove (e.g., eugenol), menthol, camphor, licorice and turmeric in a Carbopol gel base which can provide anti-inflammatory, antioxidant and antibacterial properties for gum irritation relief benefits. In one aspect, the disclosure provides a leave on oral care formulation containing a mixture of analgesic oils (e.g., oils and/or extracts) to effectively treat a consumer’s irritated gums by providing soothing relief.

The orally acceptable analgesic “leave-on” gel formulation of the disclosure comprises a mixture of oils and extracts comprising: (a) clove oil and/or eugenol (e.g., clove leaf oil and/or clove bud oil), (b) a cooling agent, e.g., selected from menthol, peppermint oil, menthyl esters, menthoxyalkanols, andp-menthane carboxamides (e.g., N-ethyl-p-menthane-3-carboxamide), and mixtures thereof; (c) camphor, (d) an antimicrobial agent, e.g., turmeric and/or curcumin extract (e.g., turmeric, white turmeric (white curcumin) and (e) licorice, in an orally acceptable gel base, the gel base comprising an anionic polymer, the formulation providing a sustained release of the mixture of analgesic oils following application. In one embodiment, the gel base comprises
a. poly(acrylic acid), e.g., carbomer homopolymer type B, e.g., Carbopol®974P NF;
b. propylene glycol;
c. nonionic surfactant selected from poloxamers, polysorbates, and mixtures thereof;
d. a source of analgesic oils and/or extracts comprising: clove oil and/or eugenol, camphor, menthol, licorice and turmeric;
e. water; and
f. optionally sweeteners, flavorings, and/or preservatives.

In other aspects, the disclosure provides methods of making such formulations and of using such formulations to alleviate tooth pain and/or gum inflammation and irritation. Without being bound by theory, it is believed that these oils and extracts are more effective as analgesics in combination with one another, perhaps because they affect different receptors. Formulation presents some challenges, as the oral cavity is essentially an aqueous environment, and the oils and extracts may be poorly soluble in water, but the structured formulations provided give stable formulations as well as effective and sustained release of the analgesic oils upon application. The formulations of the disclosure are believed to improve a user’s oral health by providing benefits with respect to freshness and antioxidant, anti-inflammatory and antimicrobial capabilities.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF FIGURES

FIG. 1 shows IL-1beta mediated PGE2 level in culture supernatants of untreated (negative control), IL-1 beta treated (positive control), IL-1 beta and Placebo gel treated, and IL-beta and Formula 1 treated tissues.

FIG. 2 shows cell viability (measured by light absorbance at 590 nm) of untreated, placebo gel treated, and Formula 1 treated tissues.

### DETAILED DESCRIPTION

The following description of the preferred embodiment(s) is merely exemplary in nature and is in no way intended to limit the disclosure, its application, or uses.

As used herein, the term “effective amount” means the quantity of extracts and/or oils required to provide an anti-sensitivity or pain-relieving effect. Tooth pain may be a result of caries, a cracked tooth, an exposed tooth root, or erosion of the enamel, as well as by gum disease, abscess, or impaction. Gum pain may be the result of bacteria in the mouth that forms plaque on the teeth or gums and eventually results in inflammation and irritation.

In some embodiments, the present disclosure provides in a first embodiment, an orally acceptable topical analgesic gel (Formulation 1) comprising an orally acceptable topical analgesic gel comprising a mixture of herbal oils and extracts comprising (a) clove oil and/or eugenol (e.g., clove leaf oil and/or clove bud oil), (b) a cooling agent (e.g., menthol), (c) camphor, (d) an antimicrobial agent, e.g., turmeric and/or curcumin extract (e.g., turmeric, white turmeric (e.g., white curcumin) and (e) licorice; in an orally acceptable gel base, the gel base comprising one or more nonionic surfactants, the formulation providing a sustained release of the mixture of analgesic oils following application to a tooth or gums.

For example, the disclosure provides e.g.,

1.1.Formulation 1 wherein the gel base provides sustained release of the herbal oils and extracts for a period of at least three, e.g., at least five minutes, e.g., at least 10 minutes, e.g., at least 20 minutes following application of the formulation to a tooth.

1.2.Formulation 1 or 1.1 wherein the mixture of herbal oils and extracts comprises 1-5% by weight of the formulation, e.g., about 2.25%.

1.3.Any of the preceding formulations wherein the ratio (wt%) of (a) to (b) to (c) is 5-1: 5 - 0.5: 5-0.5, e.g., about 3:1:1 to about 1:1:1 (e.g., 2:1:1).

1.4.Any of the preceding formulations wherein the mixture of herbal oils and extracts comprises clove oil and/or eugenol: menthol: camphor in a ratio (wt%) of 5-1: 5- 0.5 : 5-0.5, e.g., about 3:1:1 to about 1:1:1 (e.g., 2:1:1).

1.5.Any of the preceding formulations wherein the cooling agent is a TRMP8 thermoreceptor agonist, e.g., selected from peppermint oil, menthol, menthoxyalkanols (e.g., 3-(1-menthoxy)-2-methylpropane-1,2-diol, 3-(1-menthoxy)ethanol, 3-(1-menthoxy)propan-1-ol, and 3-(1-menthoxy)butan-1-ol), menthyl esters (e.g., menthyl lactate and menthyl 3-hydroxybutarate), and p-menthane 3-carboxamides (e.g., N-ethyl-p-menthane-3-carboxamide (WS-3),(1R,2S,5R)-N-(4-methoxyphenyl)-ρ-menthanecarboxamide (WS-12), (2S,5R)-N-[4-(2-Amino-2-oxoethyl)phenyl]-p-menthanecarboxamide, N-cyclopropyl-5-methyl-2-isopropylcyclohexanecarbonecarboxamide, ethyl 3-(p-menthane-3-carboxamido)acetate (WS-5), (1R,2S,5R)-N-(4-Methoxyphenyl)-ρ-menthanecarboxamide (WS-12), N-ethyl-2,2-diisopropylbutanamide (WS-27), N-cyclopropyl-5-methyl-2-isopropylcyclohexanecarboxamide, N-( 1,1-Dimethy1-2-hydroxyethyl)-2,2-diethylbutanamide (WS-116), N-(4-cyanomethylphenyl)-p-menthanecarboxamide, and N-(2-(pyridin-2-yl)ethyl)-3-p-menthanecarboxamide; for example N-ethyl-p-menthane-3-carboxamide), and mixtures thereof.

1.6. Any of the preceding formulations wherein the cooling agent is menthol.

1.7. Any of the preceding formulations further comprising an anionic polymer, wherein the anionic polymer is a polycarboxylate, e.g., selected from poly(acrylic acid)(optionally cross-linked, e.g., with polyalkenyl ethers or divinyl glycol) and optionally additionally comprising 1:4 to 4:1 copolymers of maleic anhydride or acid with another polymerizable ethylenically unsaturated monomer (e.g., methyl vinyl ether/maleic anhydride (PVM/MA) copolymer), in free or salt form.

1.8. Any of the preceding formulations wherein the gel base comprises one or more nonionic surfactants selected from: poloxamers, polysorbates, and mixtures thereof.

1.9. Any of the preceding formulations wherein the gel base comprises a nonionic surfactant selected from: nonionic triblock copolymers composed of a central hydrophobic chain of polyoxypropylene (poly(propylene oxide)) flanked by two hydrophilic chains of polyoxyethylene (poly(ethylene oxide)) (e.g., commercially available under the trade names Pluronic® or Kolliphor ®, e.g., poloxamer 407), polysorbates (e.g., polyethoxylated sorbitan esterified with fatty acids, e.g., commercially available as Alkest®, Canarcel® or Tween®, e.g., polysorbate 20), and mixtures thereof.

1.10. Any of the preceding formulations wherein the gel base has pH 6-8, e.g., wherein the gel base is approximately pH neutral.

1.11. Any of the preceding formulations wherein the gel base further comprises one or more astringents, e.g., herbal astringents, e.g., selected from Terminalia arjuna and Acacia extracts or powders.

1.12. Any of the preceding formulations wherein the gel base further comprises a soluble potassium salt, e.g., potassium nitrate.

1.13. Any of the preceding formulations wherein the gel base further comprises a small particle occlusive agent, e.g., a small particle silica or calcium carbonate, having a d₅₀ of less than 5 µm, e.g., 0.5-5 µm, e.g., a small particle synthetic amorphous silica (e.g., d₅₀ about 3-4 µm) and/or small particle precipitated calcium carbonate (e.g., d₅₀ about 0.5-3 µm).

1.14. Any of the preceding formulations wherein the mixture of analgesic oils further comprises comprising a warming agent, e.g., a TRPV1 thermoreceptor agonists, e.g., black pepper oil, ginger oil, vanilla extract, vanillyl butyl ether, capsicum tincture or mixtures of any of these, e.g., ginger oil, pepper oil or mixtures thereof.

1.15. Any of the preceding formulations wherein the mixture of herbal oils and extracts further comprises an anti-inflammatory herbal oil, e.g., a phenolic herbal oil, for example thymol.

1.16. Any of the preceding formulations wherein the gel base comprises sweeteners, e.g., saccharin, flavorings (in addition to the analgesic oils), and/or preservatives, e.g., sodium benzoate.

1.17. Any of the preceding formulation wherein the gel base comprises an amino acid (e.g., basic amino acid).

1.18. The formulation of 1.17 wherein the gel base comprises arginine (e.g., L-arginine).

1.19. Any of the preceding formulations, wherein the mixture of analgesic oils comprises (a) clove oil and/or eugenol, (b) menthol, (c) camphor, (d) turmeric and (e) licorice.

1.20. Any of the preceding formulations wherein the gel base comprises:
a. cross-linked poly(acrylic acid), e.g., carbomer homopolymer type B, e.g., Carbopol®974PNF;
b. propylene glycol;
c. nonionic surfactant selected from poloxamers (e.g., Poloxamer 407), polysorbates (e.g., Polysorbate 20), and mixtures thereof;
d. neutralizing base, e.g., sodium hydroxide; and
e. water.

For example, in one aspect the disclosure provides Formulation 1.20, an orally acceptable topical analgesic gel according to any of the preceding formulations comprising

| Carbomer homopolymer type B | 0.8-1.2%, e.g., about 1% |
|---|---|
| Propylene Glycol | 12-18%, e.g., about 15% |
| Poloxamer | 6-12%, e.g., about 9% |
| Polysorbate | 4-8%, e.g., about 6% |
| Sodium Saccharin | 0.1-0.5%, e.g., about 0.2% |
| Sodium Benzoate | 0.1-0.5%, e.g., about 0.2% |
| Base, e.g., NaOH | To adjust to pH 6-8 (e.g., about 0.28%) |
| Eugenol and/or Clove oil | 0.5-1.5%, e.g., about 1% |
| Camphor | 0.2-0.8%, e.g., about 0.5% |
| Menthol | 0.2-0.8%, e.g., about 0.5% |
| Licorice | 0.1-0.5%, e.g., about 0.2% |
| Turmeric (curcuma longa) | 0.001% - 0.0075%, e.g., about 0.004 |
| Water | 30-85% |
| Total | 100% |

In another aspect the disclosure provides Formulation 1.21, wherein the formulation is any of the preceding formulations further comprising a basic amino acid, wherein the basic amino acid is selected from arginine, lysine, citrullene, ornithine, creatine, histidine, diaminobutanoic acid, diaminoproprionic acid.

In yet another aspect the disclosure provides Formulation 1.22, wherein the formulation is any of the preceding formulations and wherein the basic amino acid (e.g., arginine) is present in an amount from 1-15%, e.g., about 1.5%, about 5%, about 8%.

In other aspects, the disclosure provides methods of making such formulations and of using such formulations to alleviate tooth pain.

For example, the disclosure provides a method of alleviating dental pain comprising administering an effective amount of a composition of any of Formulation 1, et seq. to the affected area, e.g., wherein the composition is left on the affected area following application, e.g., for at least a minute, e.g., at least 5 minutes. The disclosure further provides any of Formulation 1, et seq. for use in alleviating dental pain.

In other aspects, the disclosure provides methods of making such formulations and of using such formulations to alleviate gum irritation and/or inflammation.

For example, the disclosure provides a method of alleviating gum irritation and/or inflammation comprising administering an effective amount of a composition of any of Formulation 1, et seq. to the affected area, e.g., wherein the composition is left on the affected area following application, e.g., for at least a minute, e.g., at least 5 minutes. The disclosure further provides any of Formulation 1, et seq. for use in alleviating gum irritation and/or inflammation.

In another embodiment, the disclosure provides the use of a mixture of herbal oils and extracts comprising (a) eugenol (e.g., from clove leaf oil and/or clove bud oil), (b) a cooling agent, (c) camphor, (d) an antimicrobial agent, e.g., turmeric and/or curcumin extract (e.g., turmeric, white turmeric (e.g., white curcumin) and (e) licorice, together with an anionic polymer, in the manufacture of an orally acceptable topical analgesic gel, e.g., a gel according to any of Formulation 1, et seq., to alleviate dental pain.

In another embodiment, the disclosure provides a method of making an orally acceptable topical analgesic gel, e.g., as hereinbefore described, e.g., any of Formulation 1, et seq. the orally acceptable topical analgesic gel comprising a mixture of herbal oils and extracts comprising a) eugenol and/or clove oil (e.g., clove leaf oil and/or clove bud oil) and/or eugenol, (b) a cooling agent, and (c) camphor, (d) an antimicrobial agent, e.g., turmeric and/or curcumin extract (e.g., turmeric, white turmeric (white curcumin) and (e) licorice; together with an orally acceptable gel base comprising a cross-linked poly(acrylic acid) polymer, nonionic surfactants, a basic amino acid, and water, the method comprising first forming a water-in-oil emulsion wherein the oil phase comprises the mixture of analgesic oils, the water phase comprises the poly(acrylic acid) polymer and water, and the nonionic surfactants facilitate the emulsion formation, then adding the basic amino acid to raise the pH of the emulsion thus formed to a level sufficient to ionize the carboxyl groups on the cross-linked poly(acrylic acid) polymer, thereby forming a stable gel.

Herbal oils and extracts: In various embodiments, the disclosure provides compositions comprising a mixture of herbal oils and extracts comprising (a) eugenol and/or clove oil (e.g., clove leaf oil and/or clove bud oil); (b) a cooling agent, e.g., selected from peppermint oil, menthol, menthyl esters, menthoxyalkanols, and p-menthane 3-carboxamides), and mixtures thereof; (c) camphor; (d) turmeric and/or curcumin extract (e.g., turmeric, white turmeric (white curcumin) and (e) licorice. These herbal oils and extracts may be from natural sources or may be synthetic. Clove oil is extracted from the buds, leaf or stem of the clove plant, Syzygium aromaticum. Where clove oil is used in place of eugenol, the amount used may be adjusted, so that the amount of eugenol (typically 80-90% of the clove oil) is constant. Camphor may be extracted from plants, e.g., from laurel or rosemary, or synthetically produced, e.g., from oil of turpentine. Cooling agents are known in the art and are described, e.g., in Leffingwell, “Cooling Ingredients and Their Mechanisms of Action”, Chapter 65, from Barel, et al. (Eds), Handbook of Cosmetic Science and Technology, 3^{rd} ed. Informa Healthcare (2009), the contents of which are incorporated herein by reference. Cooling agents include natural or synthetic TRMP8 thermoreceptor agonists, for example, menthol, which is the dominant component of peppermint oil, and its various derivatives, e.g., compounds having a ρ-menthane (1-methyl-4-isopropyl-cyclohexyl) moiety, for example menthyl esters, menthoxyalkanols, and p-menthane 3-carboxamides. The analgesic oils may in some embodiments additionally comprise one or more warming agents, e.g., TRPV1 thermoreceptor agonists, e.g., black pepper oil, ginger oil, vanilla extract, vanillyl butyl ether, capsicum tincture, or mixtures thereof, for example in an amount of 0.1-2%, e.g., ca. 0.5% ginger oil and 0.5% black pepper oil. The herbal oils and extracts may in some embodiments additionally comprise one or more anti-inflammatory herbal oils, e.g., selected from the phenolic herbal oil, for example thymol, for example in an amount of 0.1-2%, e.g., ca. 0.5% thymol.

The gels of the disclosure, e.g., any of Formulation 1.0 et seq, in various embodiments utilize anionic polymers, which tend to stick to the soft and hard surfaces of the oral cavity, and provide targeted sustained delivery of the analgesic oils. Carbomers or polyacrylates, for example the various Carbopol® products, are cross-linked poly(acrylic acid) polymers, and may in certain embodiments form the principal structurant for the gels. For example, the gel may comprise cross-linked poly(acrylic acid), e.g., carbomer homopolymer type B, for example Carbopol®974PNF. These polymers have low viscosity at low pH, permitting mixing of the various components of the gel, and formation of an emulsion or suspension of the analgesic oils, but when the pH is raised by addition of a basic material, the carboxyl moieties ionize and repel one another, causing the polymer to swell and the viscosity to increase. The oil droplets trapped in the matrix remain stably suspended in the formulation, and are then released upon application. Thus the polyacrylate component is typically added in the form of the free acid and then partially or fully neutralized with a suitable base in the final formulation to form water soluble alkali metal (e.g., potassium and sodium) or ammonium salts. The polyacrylate is provided in an amount sufficient to provide a viscous gel in the final formulation, e.g., 0.5-3%, for example 1-1.5%. The compositions of the disclosure may in some embodiments comprise additional polymers, for example 1:4 to 4:1 copolymers of maleic anhydride or acid with another polymerizable ethylenically unsaturated monomer (e.g., methyl vinyl ether/maleic anhydride (PVM/MA) copolymer), e.g., having a molecular weight (M.W.) of about 30,000 to about 1,000,000, most preferably about 300,000 to about 800,000. Methyl vinyl ether/maleic anhydride (PVM/MA) copolymers include the Gantrez® product line, e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805.

In some embodiments any of Formulation 1.0 et seq comprises an additional anionic polymers present in amounts ranging from about 0.05 to about 3% by weight. Finally, in some embodiments, the compositions comprise additional thickening agents, for example, polyvinyl pyrrolidone (PVP, e.g. Plasdone® S-630), and/or silica thickeners, which form polymeric structures or gels in aqueous media, may be present. Note that these silica thickeners are physically and functionally distinct from particulate silica abrasives often used in dentifrice formulations, as the silica thickeners are very finely divided and provide little or no abrasive action. Other thickening agents may include carboxyvinyl polymers, carrageenan, hydroxyethyl cellulose and/or water soluble salts of cellulose ethers such as sodium carboxymethyl cellulose and sodium carboxymethyl hydroxyethyl cellulose. Natural gums such as karaya, gum arabic, and gum tragacanth may also be incorporated. Colloidal magnesium aluminum silicate can also be used as component of the thickening composition to further improve the composition’s texture. In certain embodiments, thickening agents in addition to the polycarboxylate may be found in an amount of about 0.5% to about 10.0% by weight of the total composition are used.

In some embodiments, any of Formulation 1.0 et seq comprises a small particle occlusive agent, capable of plugging the dentinal tubules and reducing sensitivity of the teeth. The small particle occlusive agent may, for example, be a small particle silica or calcium carbonate, having a d₅₀ of less than 5 µm, e.g., 0.5-5 µm, e.g. small particle synthetic amorphous silica (d₅₀ about 3-4 µm) and/or small particle precipitated calcium carbonate (d₅₀ about 0.5-3 µm). For example, commercially available Sorbosil AC43 silica has a d₅₀ of 3.95 µm. The d₅₀ is measured using particle size measuring techniques as known in the art. For example, particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silica hydrogel, for example. It will be understood that other methods of measuring particle size are known in the art, and based on the disclosure set forth herein, the skilled artisan will understand how to calculate median particle size, mean particle size, and/or particle size distribution of particles in the present disclosure.

In certain embodiments, e.g., any of Formulation 1.0 et seq, the emulsion of the oil/extract in the gels of the disclosure is facilitated by surfactants. In some embodiments, the surfactants are nonionic surfactants. Illustrative nonionic surfactants that can be used in the compositions of the disclosure include compounds produced by the condensation of alkylene oxide groups (generally hydrophilic) with an organic hydrophobic compound which may be aliphatic or alkylaromatic. Examples of nonionic surfactants include, but are not limited to, the poloxamers (e.g., nonionic triblock copolymers composed of a central hydrophobic chain of polyoxypropylene (poly(propylene oxide)) flanked by two hydrophilic chains of polyoxyethylene (poly(ethylene oxide)), for example commercially available under the trade names Pluronic® or Kolliphor ®, e.g, poloxamer 407), polysorbates (polyethoxylated sorbitan esterified with fatty acids, for example commercially available as Alkest®, Canarcel® or Tween®, e.g. polysorbate 20) and mixtures thereof. Other nonionic surfactants which may be used include polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. The surfactant or mixtures of compatible surfactants can be present in the compositions of the present disclosure in an amount sufficient to permit formation of an oil-in water emulsion between (i) the water and other hydrophilic components of the gel and (ii) the analgesic oils, e.g., in about 0.1% to about 20.0%, for example about 5% to about 20% by weight of the total composition.

In some embodiments, e.g., any of Formulation 1.0 et seq, the viscosity of the compositions of the disclosure is greater than about 1,000 centipoise (cPs) and less than about 900,000 cPs, in a more specific embodiment greater than about 10,000 cP and less than about 100,000 cPs, in a more specific embodiment greater than 50,000 cPs and less than 900,000 cPs, and in an even more specific embodiment from between about 200,000 cPs to about 600,000 cPs.

In some aspects, e.g., any of Formulation 1.0 et seq, can comprise a basic amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrulline, ornithine, creatine, histidine, diaminobutanoic acid, diaminoproprionic acid, salts thereof or combinations thereof. In a particular embodiment, the basic amino acids are selected from arginine, citrulline, and ornithine. In certain embodiments, the basic amino acid is arginine, for example, L-arginine, or a salt thereof.

In another aspect, the compositions of the invention (e.g., any of Formulations 1.0 et seq) can further comprise one or more neutral amino acid, which can include, but is not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the disclosure, e.g., any of Formulation 1.0 et seq, may optionally comprise other components, for example humectants, e.g., propylene glycol and/or glycerin, flavoring agents (in addition to the herbal oils/extracts), sweetening agents, e.g., sodium saccharin, preservatives, e.g., sodium benzoate, basifying agents, e.g., sodium hydroxide, and/or coloring agents.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

Embodiments of the present disclosure are further described in the following examples. The examples are merely illustrative and do not in any way limit the scope of the disclosure as described and claimed.

### EXAMPLES

### Example 1

Gels used in the Examples are prepared with the following ingredients and process:

TABLE 1

Formula 1

| Carbomer 974P NF | 0.8-1.2%, e.g., about 1% |
|---|---|
| Propylene Glycol | 12-18%, e.g., about 15% |
| Poloxamer | 6-12%, e.g., about 9% |
| Polysorbate | 4-8%, e.g., about 6% |
| Sodium Saccharin | 0.1-0.5%, e.g., about 0.2% |
| Sodium Benzoate | 0.1-0.5%, e.g., about 0.2% |
| Base, e.g., NaOH | To adjust to pH 6-8 (e.g., about 0.28%) |
| Clove oil (incl. clove leaf oil and clove bud oil) | 0.5-1.5%, e.g., about 1% |
| Camphor | 0.2-0.8%, e.g., about 0.5% |
| Menthol | 0.2-0.8%, e.g., about 0.5% |
| Licorice | 0.1-0.5%, e.g., about 0.2% |
| Turmeric (curcuma longa) | 0.001% - 0.0075%, e.g., about 0.004 |
| Water | q.s. (e.g., 50% - 80%) |
| Total | 100% |

The gel base formulation for the Examples is made as follows:
1. In this step sodium saccharin, sodium benzoate, licorice and Turmeric are mixed together in Polysorbate 20, Poloxamer 407 and water.
2. In this step the base is prepared with Carbomer 974P NP in Propylene glycol and water. Then mixture of step 1 is mixed with step 2.
3. In this step clove leaf oil, clove flower bud oil, Camphor and Menthol are mixed together and then added to the base made in step 2.
4. Finally, the pH of the total gel base formulation is adjusted with sodium hydroxide 50%.

### Example 2 - Anti-Inflammatory Assays

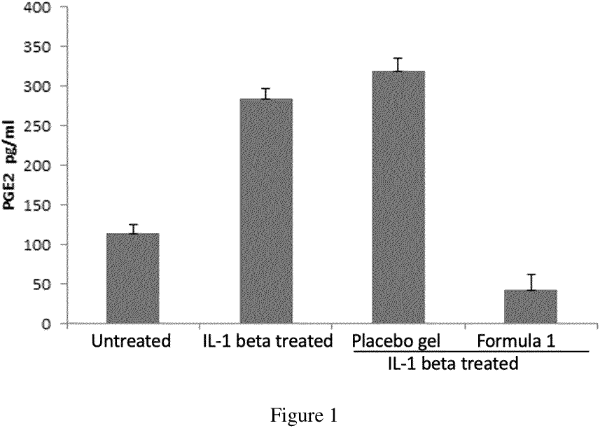

A placebo gel and a gel base formulation of Example 1 (Formula 1) are made into slurries and prepared in water and applied topically in an in vitro gingival tissue model. Treated tissues are fed with tissue culture media, incubated overnight in their respective incubators and supernatants are collected and assessed by a PGE2 ELISA assay. The results are shown in FIG. 1. In-vitro studies demonstrate that when compared to placebo gel (base) treated gingival tissues, Formula 1 treated tissues demonstrate significant reduction in IL-1beta mediated PGE2 inflammatory biomarker in culture supernatants.

### Example 3 - Cytotoxicity Assays

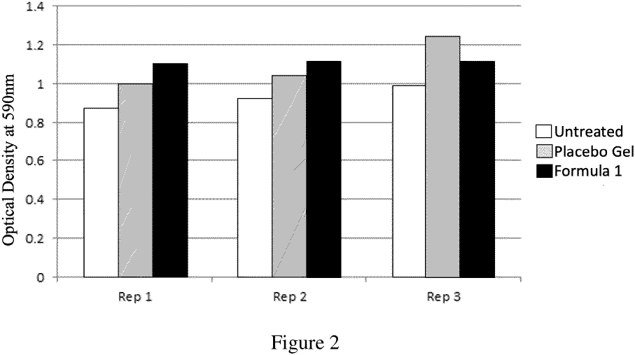

Gingival tissues undergo topical treatment with the placebo gel or a slurry of the gel base formulation (Formula 1) described in Example 1. Tissues undergo incubation in an overnight incubator. Tissues undergo staining with MTT dye and dye assay absorbance is captured. MTT is used to assess cell viability as a function of redox potential. Actively respiring cells convert the water-soluble MTT to an insoluble purple formazan. The experiments are repeated three times. The results are shown in FIG. 2. The data demonstrates that treatment with the placebo gel or a slurry of Formula 1 have similar absorbance which suggests that tissue health/viability is maintained in treated tissues.

### Example 4 - Antioxidant Activity

The study is designed to determine the antioxidant potential of placebo and the gel base formulation of Example 1 in terms of the DPPH free radical scavenging activity values reported as a percentage of antioxidant activity. 1,1-diphenyl-2-picrylhydrazyl (DPPH) is a free radical which has an unpaired valence electron at one atom of its nitrogen bridge. In its oxidized form, DPPH radical has an absorbance maximum centered on 517 nm exhibiting purple-blue color. In the presence of an antioxidant molecule, DPPH radical decolorizes.

In general, a placebo gel and a gel base formulation of Example 1 (Formula 1) are made into slurries and prepared in water and supernatant is collected. DPPH solution (prepared in ethanol) is then added to this supernatant and incubated in dark. The intensity of blue-purple color is measured spectrophotometrically at 517 nm.The intensity of blue-purple color is inversely proportional to the antioxidant potential.

The antioxidant potential is directly proportional to the reduced intensity of purple DPPH solution. It is calculated by conducting measurements spectrophotometrically at 517 nm in the presence of test compounds. Data suggests that the gel base formulation of Example 1 exhibits acceptable antioxidant activity which is significantly higher than the placebo gel:

TABLE 2

DPPH Assay

| Sample | % Antioxidant Activity* |
|---|---|
| Internal positive control | 99.85% |
| Placebo gel | 48.25% |
| Formula 1 | 72.75% |

* The color intensity of the test sample is compared to the color intensity of vehicle control (Ethanol)

### Example 5 - In Vitro Plaque Glycolysis Assay

Bacterial biofilms are important mediators of human disease, especially within the oral cavity. Disruption of pre-formed oral plaque biofilms is a critical step to effective oral cleansing. This assay is designed to quantify the ability of formulations to reduce the bacterial metabolic activity of multi-species biofilms grown from saliva on an artificial tooth surface by single treatment, either through antibacterial or anti-metabolic activity. Data suggests that the gel base formulation of Example 1 exhibits a significantly better antibacterial activity compared to its placebo gel as measured by change in pH. “Significantly better” antibacterial activity refers to less bacterial growth after treatment.

As antibacterial activity can cause a decrease in pH in the oral cavity due to metabolic activity of growing bacteria, a decrease in Δ pH is believed to be indicative of lower antibacterial activity:

TABLE 3

| In vitro Plaque Glycolysis | Mean Δ pH |
|---|---|
| Internal negative control* | 2.03 |
| Placebo gel | 1.67 |
| Formula 1 | 1.47 |

* “Internal negative control” is a commercial toothpaste that is not believed to contain any of: eugenol, turmeric, licorice or camphor. Placebo gel does not include active.

1. An orally acceptable topical analgesic gel comprising the following herbal oils and extracts:
(a) clove oil and/or eugenol,
(b) a cooling agent;
(c) camphor,
(d) an antimicrobial agent, and
(e) licorice,
in an orally acceptable gel base, the gel base comprising one or more nonionic surfactants, the formulation providing a sustained release of the mixture of herbal oils and extracts following application wherein the gel base provides controlled release of the mixture of herbal oils and extracts following application to the gums or teeth.

2. The analgesic gel of claim 1, wherein the mixture of herbal oils and extracts comprises 1-5% by weight of the formulation, e.g., about 2.25%.

3. The analgesic gel of claim 1, wherein the ratio (wt%) of (a) to (b) to (c) is 5-1: 5 - 0.5: 5-0.5.

4. The analgesic gel of claim 1, wherein the mixture of herbal oils and extracts comprises clove oil and/or eugenol: menthol: camphor in a ratio (wt%) of 5-1: 5- 0.5 : 5-0.5.

5. The analgesic gel of claim 1, wherein the cooling agent is a TRMP8 thermoreceptor agonist selected from peppermint oil, menthol, menthoxyalkanols, menthyl esters, p-menthane 3-carboxamides and mixtures thereof.

6. The analgesic gel of claim 1, wherein the cooling agent is menthol.

7. The analgesic gel of claim 1 further comprising an anionic polymer, wherein the anionic polymer is a polycarboxylate.

8. The analgesic gel of claim 1, wherein the nonionic surfactants in the gel base are selected from: poloxamers, polysorbates, and mixtures thereof.

9. The analgesic gel of claim 1, wherein the gel base comprises a nonionic surfactant selected from: nonionic triblock copolymers composed of a central hydrophobic chain of polyoxypropylene (poly(propylene oxide)) flanked by two hydrophilic chains of polyoxyethylene (poly(ethylene oxide)), polysorbates, and mixtures thereof.

10. The analgesic gel of claim 1, wherein the mixture of herbal oils and extracts further comprises comprising a warming agent.

11. The analgesic gel of claim 1, wherein the gel base comprises an amino acid.

12. The analgesic gel of claim 11, wherein the gel base comprises arginine.

13. The analgesic gel of claim 1, wherein the gel base comprises:
a. carbomer homopolymer type B;
b. propylene glycol;
c. nonionic surfactant selected from poloxamers, polysorbates, and mixtures thereof;
d. sodium hydroxide; and
e. water.

14. The analgesic gel of claim 1, wherein the mixture of herbal oils and extracts comprises (a) clove oil and/or eugenol, (b) menthol, (c) camphor, (d) turmeric and (e) licorice.

15. A method of alleviating dental pain comprising administering an effective amount of an analgesic gel according to claim 1 to the affected area, wherein the composition is left on the affected area following application for at least sixty seconds.

16. A method of alleviating gum irritation and/or inflammation comprising administering an effective amount of an analgesic gel according to claim 1 to the affected area, wherein the composition is left on the affected area following application for at least a minute.

17. (canceled)
